(19) United States
(12) Patent Application Publication
(10) Pub. No.: US 2023/0191081 A1
(43) Pub. Date: Jun. 22, 2023

(54) CATHETER DEVICES AND METHODS OF USE

(71) Applicant: Piraeus Medical, Inc., Rochester, MN (US)

(72) Inventor: Jared Hutar, Cromwell, MN (US)

(21) Appl. No.: 18/064,240

(22) Filed: Dec. 9, 2022

Related U.S. Application Data
(60) Provisional application No. 63/290,545, filed on Dec. 16, 2021.

(51) Int. Cl. A61M 25/00; A61B 17/22

(52) U.S. Cl. CPC A61M 25/0045; A61B 17/22; A61M 25/0053; A61B 2017/22079; A61B 2017/00942

(57) ABSTRACT

The disclosure includes a catheter comprising a proximal end, a distal end located opposite the proximal end, an outer surface extending between the proximal end and the distal end, and an inner surface located opposite the outer surface, wherein the inner surface is configured to also extend between the proximal end and the distal end. In some embodiments, each of the outer and inner surfaces is coated in a hydrophilic coating. The hydrophilic coating may be configured to reduce surface tension and increase the lubricity of the catheter.

### CROSS-REFERENCE TO RELATED APPLICATIONS

The entire contents of the following application are incorporated by reference herein: U.S. Provisional Patent Application No. 63/290,545; filed Dec. 16, 2021; and entitled CATHETER DEVICES AND METHODS OF USE.

### SUMMARY

The disclosure includes a catheter comprising a proximal end, a distal end located opposite the proximal end, a working lumen extending between the proximal end and the distal end, an outer surface extending between the proximal end and the distal end, the outer surface defining an outer diameter, and an inner surface extending between the proximal end and the distal end, the inner surface defining an inner diameter and located opposite the outer surface. In some embodiments, the catheter includes a first hydrophilic coating and a second hydrophilic coating. The first hydrophilic coating may be located on the outer surface and may be configured to reduce surface friction and increase lubricity of the outer surface. The second hydrophilic coating may be located on the inner surface and may be configured to reduce surface friction and increase lubricity of the inner surface.

In some embodiments, the working lumen is configured to receive a secondary device comprising at least one of a guidewire, a microcatheter, an intermediate catheter, and a stent retriever. The catheter may comprise a device wall including at least one polymer coupled to a catheter reinforcement structure. In some embodiments, the catheter reinforcement structure comprises a braid and coil reinforcement structure. The braid and coil reinforcement structure may comprise a coil defining a pitch smaller than 0.03 inches. The at least one polymer may be configured to provide at least one of flexibility and structural support to the catheter. In some embodiments, the device wall is located between the first hydrophilic coating and the second hydrophilic coating.

At least one of the first hydrophilic coating and the second hydrophilic coating may comprise a substantially smooth surface. In some embodiments, at least one of the first hydrophilic coating and the second hydrophilic coating comprises a ribbed surface. In some embodiments, the first hydrophilic coating extends between the proximal end and the distal end of the catheter. The first hydrophilic coating may extend along a surface extending between the proximal end and the distal end of the catheter. In some embodiments, the second hydrophilic coating extends between the proximal end and the distal end of the catheter. The second hydrophilic coating may extend along a surface extending between the proximal end and the distal end of the catheter.

In some embodiments, the outer diameter of the catheter is about 0.111 inches, and the inner diameter of the catheter is about 0.100 inches. The outer diameter of the catheter may be about 0.098 inches, and the inner diameter of the catheter may be about 0.088 inches.

The disclosure includes a method of using a catheter, the method comprising inserting the catheter into a patient's vasculature, wherein the catheter includes a proximal end and a distal end located opposite the proximal end, advancing the catheter through the patient's vasculature toward a vascular lesion, and advancing the catheter to a location selected from the group consisting of a first location and a second location. In some embodiments, the first location is within a first predetermined distance from the vascular lesion, and the second location is within a second predetermined distance from the vascular lesion. When the catheter is in the first location, the catheter may be able to aspirate the vascular lesion, and when the catheter is in the second location, the catheter may be unable to aspirate the vascular lesion. In some embodiments, when the catheter is in the first location, the method further comprises aspirating the vascular lesion with the catheter. When the catheter is in the second location, the method may further comprise advancing a secondary device through the catheter toward the first location. In some embodiments, when the secondary device is in the first location, the method further comprises aspirating the vascular lesion with the secondary device. The vascular lesion may comprise an occlusion located in a patient's brain.

### BRIEF DESCRIPTION OF THE FIGURES

FIG. 1 illustrates a perspective view of a catheter, according to some embodiments.

FIGS. 2A and 2B illustrate a catheter, according to some embodiments.

FIGS. 3 and 4 illustrate cross-sections of a catheter, according to some embodiments.

FIG. 5 illustrates a catheter, according to some embodiments.

FIG. 6 illustrates a catheter including various elements, according to some embodiments.

FIGS. 7 and 8 illustrate flow charts explaining the processes of applying hydrophilic coatings, according to some embodiments.

### DETAILED DESCRIPTION

Although certain embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be embodied as integrated components or as separate components.

To compare various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The invention generally relates to medical devices and methods of use. Embodiments of the invention include devices and methods for performing thrombectomy or embolectomy in a patient. Acute Ischemic Stroke (AIS) can be caused by thrombus, embolus, or other occlusions in regions of the internal carotid artery (ICA) such as the Petrous part, Cavernous part, or Cerebral part. Approaches for performing thrombectomy or embolectomy to treat AIS include positioning a device—such as an aspiration catheter, a balloon guiding catheter, or other devices—in the carotid artery at a location upstream from the occlusion, typically at a proximal location in the artery such as the cervical part. Navigation to the proximal location can be difficult due to the tortuous nature and small vessel size of the vasculature usually involved in an AIS.

Traditional approaches for treating AIS and lesions in other areas of the body involve the use of several devices of varying sizes and stiffnesses to strike the right balance of size and flexibility for navigation, trackability, and aspiration power. As a result, thrombectomy procedures can take a more significant amount of time as the physician uses trial and error to determine which device/combination of devices will reach and remove the occlusion. In a situation where “time is brain,” reducing the amount of time required to remove the thrombus, embolus, or another occlusion is crucial to achieving the best possible outcome for the patient.

There is a continuing need for improved devices and methods for mechanical revascularization such as thrombectomy and embolectomy in the ICA and other vasculature. In particular, there is a need for devices and methods that provide enhanced efficacy and efficiency of treatment.

### COMPONENT INDEX

10—catheter
12—proximal end
14—distal end
16—outer surface
18—inner surface
20—outer diameter
22—inner diameter
24a—first hydrophilic coating
24b—second hydrophilic coating
26—device wall
28—at least one polymer
30—catheter reinforcement structure
32—hub

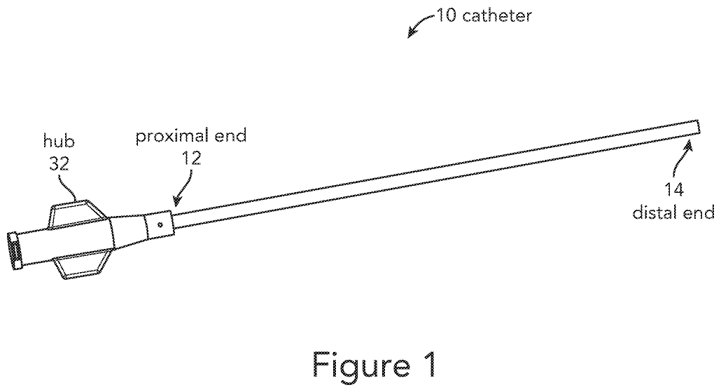

FIG. 1 illustrates a perspective view of a catheter 10, according to some embodiments. As shown, the catheter 10 may include a proximal end 12 and a distal end 14 located opposite the proximal end 12. The catheter 10 may also include a hub 32 located at the proximal end 12. The catheter 10 will be described in greater detail throughout this disclosure.

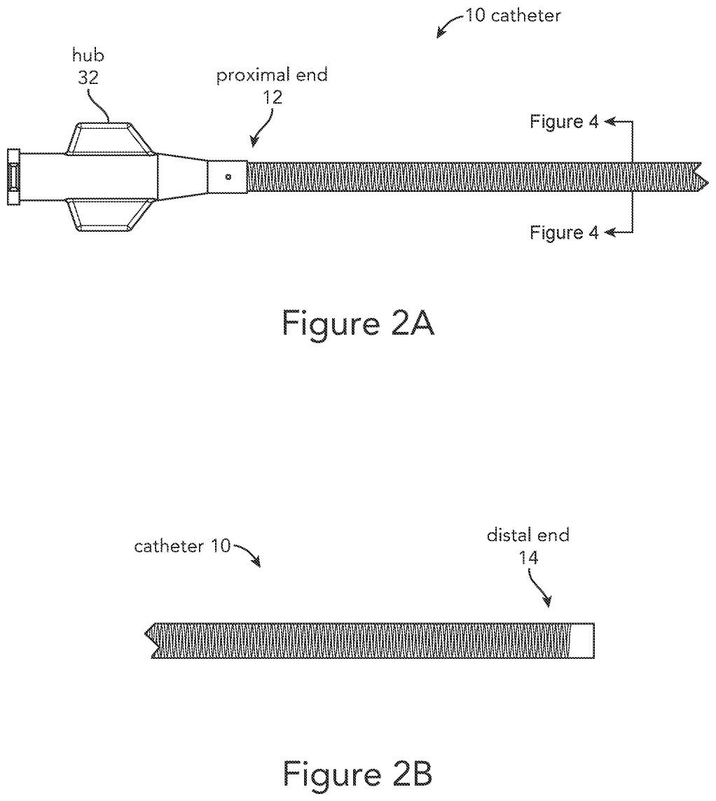

FIGS. 2A and 2B illustrate side views of the catheter 10, according to some embodiments. As mentioned with reference to FIG. 1, the catheter 10 may include a proximal end 12 with a hub 32 and a distal end 14, as illustrated in FIGS. 2A and 2B, respectively. The catheter 10 may be sized and configured to receive a secondary device, such as a guidewire, a microcatheter, an intermediate catheter, a stent retriever, and any number of other suitable devices. For example, during a procedure such as a thrombectomy, the catheter 10 may be inserted into the patient first in an initial attempt to track the catheter 10 distally within the anatomy to a surface of a clot. If the catheter 10 successfully tracks the surface of the clot, an aspiration force may be applied to the catheter 10, thereby removing the clot through the catheter 10. If the catheter 10 is unsuccessful in tracking the clot's surface, the catheter 10 may still serve as a guide or support catheter to help deliver the secondary device through the hub 32 of the catheter 10 to the surface of the clot so that the secondary device may remove the clot. The possibility of using only a single device (i.e., the catheter 10) to remove the clot allows for procedures to be more efficient than current procedure practices, which often involve several steps of introducing and removing several devices. In some embodiments, the secondary device is received by a working lumen of the catheter 10, wherein the working lumen extends between the proximal end 12 and the distal end 14 of the catheter 10.

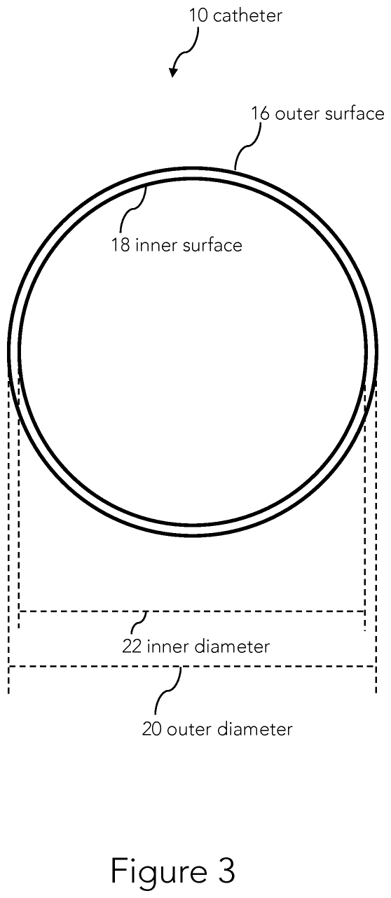

FIG. 3 illustrates a cross-section of the catheter 10. As shown, the catheter 10 may include an outer surface 16 defining an outer diameter 20 and an inner surface 18 defining an inner diameter 22. The outer diameter 20 and inner diameter 22 may each define a broad range of dimensions, including, for example, 0.111 inches for the outer diameter 20 and 0.100 inches for the inner diameter 22. In some embodiments, the outer diameter 20 measures about 0.098 inches, and the inner diameter 22 measures about 0.088 inches. It should be noted that these dimensions are included as examples only and intended to be non-limiting in scope. The outer and inner diameters 20, 22 of the catheter 10 may define dimensions not explicitly listed in this disclosure.

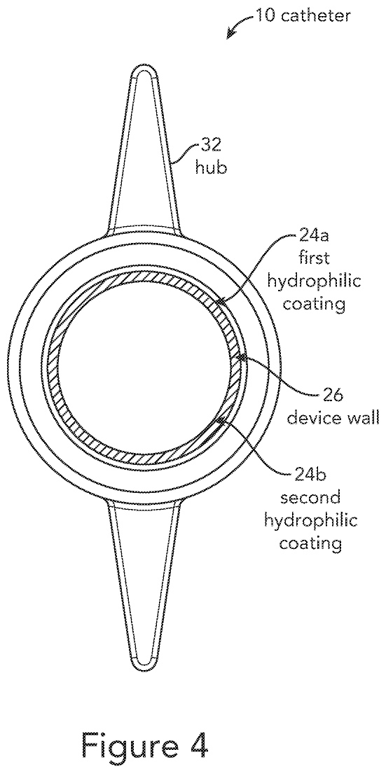

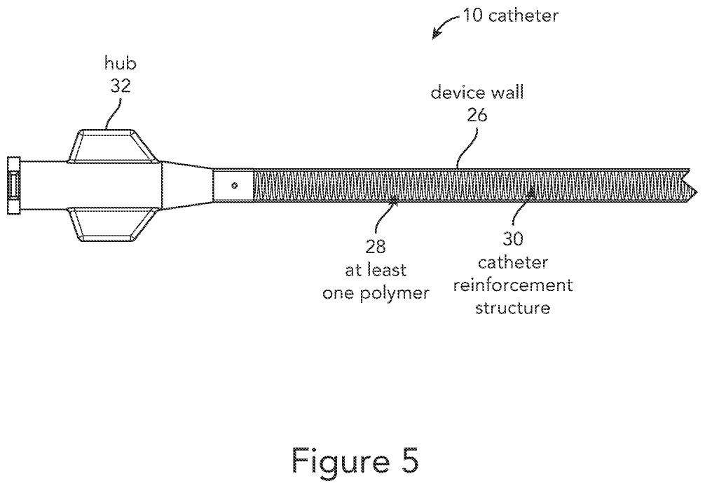

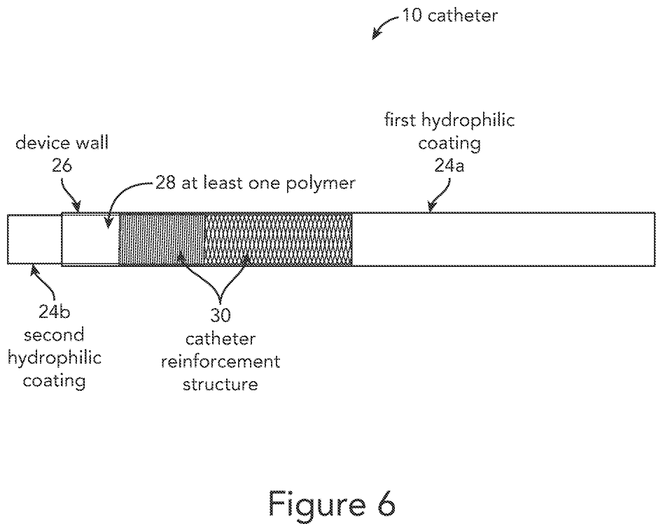

As indicated in FIG. 2A, FIG. 4 illustrates a cross-section of the catheter 10, including the hub 32. In some embodiments, as shown in FIG. 4, the catheter 10 comprises a device wall 26. The device wall 26 will be discussed further regarding FIGS. 5 and 6. The catheter 10 may also include a first hydrophilic coating 24a and a second hydrophilic coating 24b. In some embodiments, the first hydrophilic coating 24a is located on the outer surface 16 of the catheter 10, and the second hydrophilic coating 24b is located on the inner surface 18 of the catheter 10. As such, the device wall 26 may be located between the first hydrophilic coating 24a and the second hydrophilic coating 24b.

Each of the first hydrophilic coating 24a and the second hydrophilic coating 24b may extend along a surface extending between the proximal end 12 and the distal end 14 of the catheter 10. In some embodiments, the surface extends substantially an entire length of the catheter 10. The surface may extend less than a full length, such as 50%, 25%, or 10% of the entire length. In some embodiments, the first and second hydrophilic coatings 24a, 24b are configured to cover a distalmost portion, such as 15 centimeters, of the catheter 10. It should be noted that the first and second hydrophilic coatings 24a, 24b may be configured to cover any size portion of the catheter 10. It should also be noted that each of the first and second hydrophilic coatings 24a, 24b does not necessarily define the same length, though they may each define the same length.

In some embodiments, the first hydrophilic coating 24a and the second hydrophilic coating 24b comprise the same material and thickness. The thickness of the first and second hydrophilic coatings 24a, 24b may be between 0.0001 and 0.001 inches. It should be noted that the term “hydrophilic coating” is used to refer to any general type of lubricious coating that reduces friction and increases trackability of the catheter 10, including hydrophilic coatings, silicone coatings, PTFE dust, and any other suitable lubricants. The hydrophilic coating may comprise HYDAK®, produced by Biocoat® Inc. In some embodiments, the hydrophilic coatings 24a, 24b allow the device wall 26 to be thinner than a traditional device wall while also improving the performance of the catheter 10. In some embodiments, the device wall 26 defines a thickness between 0.001 and 0.04 inches. It should be noted that any one or multiple of the first hydrophilic coating 24a, the second hydrophilic coating 24b, and the device wall 26 may define a thickness other than the specific dimensions listed in this disclosure. The dimensions included here are intended to be non-limiting examples.

Referring to FIG. 5, the catheter 10 is shown, including the device wall 26. In some embodiments, as illustrated in FIG. 5, the device wall 26 comprises at least one polymer 28 and a catheter reinforcement structure 30. The catheter reinforcement structure 30 may comprise a metallic braid and coil structure, with the at least one polymer 28 filling any space within the braid and coil structure. The at least one polymer 28 may also cover the catheter reinforcement structure 30, including both exterior and interior portions of the catheter reinforcement structure 30. In general, the device wall 26 may be thought of as similar to the structure of a garden hose, with a layer of hydrophilic coating 24a, 24b on the inner and outer surfaces of the device wall 26. In some embodiments, the catheter reinforcement structure 30 is configured to provide stiffness to a proximal portion of the catheter 10 and flexibility to a distal portion of the catheter 10. Accordingly, the amount, coil tightness, and/or composition of the catheter reinforcement structure 30 may vary depending on the location along the length of the catheter 10 to vary the amount of stiffness and/or flexibility imparted by the catheter reinforcement structure 30.

FIG. 6 is similar to FIG. 5 in that it illustrates another view of the catheter 10, including the various layers of materials. Included in FIG. 6 are the first hydrophilic coating 24a, the second hydrophilic coating 24b, the device wall 26, the at least one polymer 28, and the catheter reinforcement structure 30. As discussed above, the device wall 26 may have a structure similar to that of a garden hose, where the at least one polymer 28 and the catheter reinforcement structure 30 meld together, with the first hydrophilic coating 24a located on the outer surface and the second hydrophilic coating 24b located on the inner surface.

In some embodiments, the device wall 26 has a “sandwich” structure comprising two layers of the at least one polymer 28, with the catheter reinforcement structure 30 between the polymer layers 28. As previously discussed, the catheter reinforcement structure 30 may comprise a braid and coil structure. The catheter reinforcement structure 30 may include individual coil and braid structures, as indicated by the different appearances of the catheter reinforcement structure 30 in FIG. 6. For example, the coil structure may be represented by the portion of the catheter reinforcement structure 30 to the left in FIG. 6, while the braid structure may be represented by the portion of the catheter reinforcement structure 30 to the right in FIG. 6. In some embodiments, the “sandwich” style device wall 26 comprises an inner layer of at least one polymer 28, the coil structure on top of the inner polymer layer, the braid structure on top of the coil structure, and an outer layer of at least one polymer 28. In the “sandwich” style, the device wall 26 may also include the first hydrophilic coating 24a and the second hydrophilic coating 24b.

In some embodiments, the “sandwich” style device wall 26 allows for a more open coil pitch in the coil structure, thereby enabling the catheter 10 to be softer than an embodiment where the coil structure has a tighter or more closed pitch. A softer and more flexible catheter 10 can be desirable for certain uses, such as when navigating tortuous anatomy, to give the user (i.e., a medical practitioner) more freedom to move the device at different angles. This “sandwich” style may also provide benefits from a manufacturing standpoint, as a more open coil pitch may be easier to produce with a larger margin of error than a closed pitch.

However, there may also be benefits to a device wall 26 comprising a tighter pitch coil structure. For example, in an embodiment where the catheter reinforcement structure 30 includes a coil defining a pitch smaller than 0.03 inches, the second hydrophilic coating 24b may be provided with a substantially solid and ribbed surface to adhere to. In this sense, the second hydrophilic coating 24b may be thought of as having a textured, or “ribbed,” surface. In comparison, the first hydrophilic coating 24a may be thought of as having a substantially smooth surface. In some embodiments, to ensure a sufficiently solid inner surface 18 of the catheter 10, the coil comprises a 0.002 inch round coil with a 0.004 inch pitch. A tighter pitch coil may be better for facilitating lubricity of the inner surface 18 of the catheter 10. In some embodiments, a coil with a pitch less than 0.025 inches is desirable. A sufficiently tight-pitch coil in the catheter reinforcement structure 30, combined with the second hydrophilic coating 24b on the inner surface 18 of the catheter 10, may provide enough lubricity to replace the need for a liner, such as a PTFE liner, which is traditionally used in catheter construction.

Regardless of the “style” of device wall 26 used (e.g., “sandwich” or tight-pitch coil), the use of a first and second hydrophilic coating 24a, 24b on the catheter 10 may allow for a thinner, more flexible device wall 26, as compared to other types of catheter walls without inner and outer coatings.

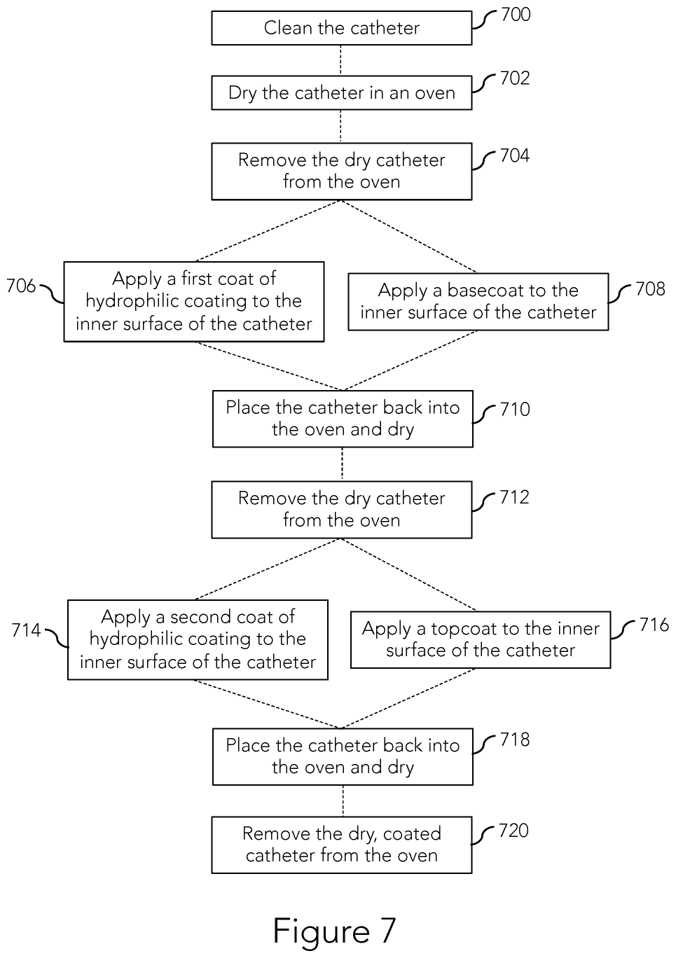

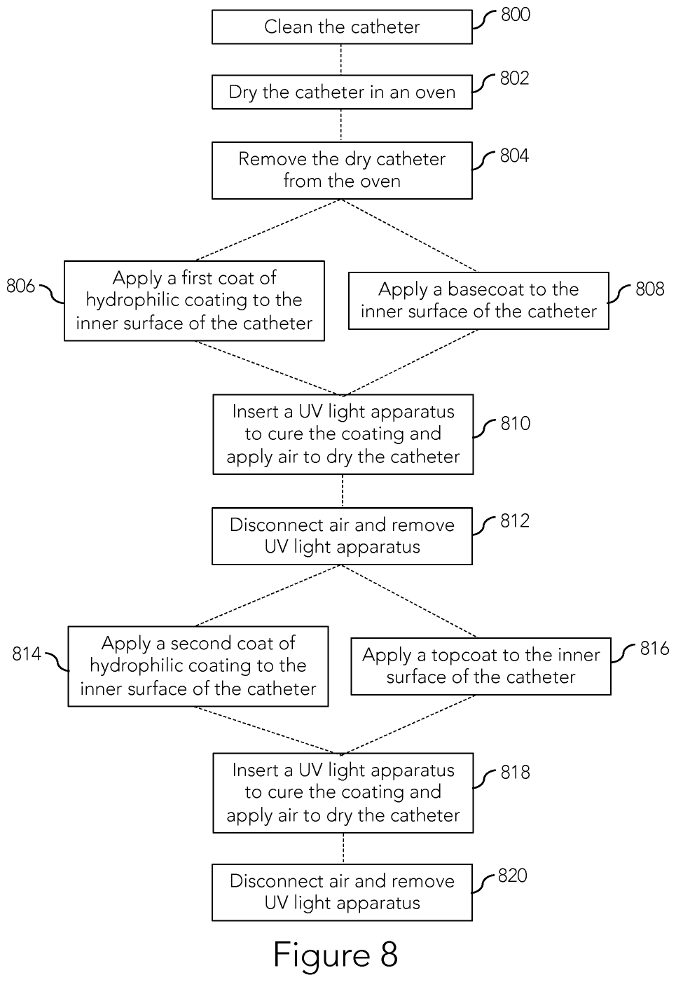

FIGS. 7 and 8 include flowcharts illustrating different processes of coating and curing an inner surface of a catheter. The “heat curing” process shown in FIG. 7 starts with cleaning the catheter, at step 700. In some embodiments, cleaning the catheter includes flushing the catheter with purified water, isopropyl alcohol (“IPA”), a mix of IPA and water, or some other suitable cleansing fluid. The next step is to dry the catheter in an oven, at step 702. The drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. The process continues with step 704: remove the dry catheter from the oven.

Next, the process can continue in one of two possible steps. One option is to apply a first coat of hydrophilic coating to the inner surface of the catheter, shown at step 706. Alternatively, a basecoat may be applied to the inner surface of the catheter, at step 708. Both steps 706 and 708 may use positive or negative pressure to fill the catheter with either the hydrophilic coating (step 706) or the basecoat (step 708). The catheter may be filled with the relevant coating material from either end of the catheter body. In some embodiments, the relevant coating material substantially continuously flows through the catheter for a predetermined amount of time to ensure an adequate amount of coating is applied. The relevant coating material may dwell within the catheter, rather than flow through, for a predetermined amount of time.

After either step 706 or step 708, the process may continue to place the catheter back into the oven to dry, at step 710. Similar to the first drying step (i.e., step 702), step 710 may involve placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. Step 710 may be considered a “heat curing” step, as heat is used to dry (i.e., cure) the coating. Next, the positive or negative pressure source is disconnected and the dry catheter is removed from the oven, at step 712.

At this point, the process again diverges into two different options. One is to apply a second coat of hydrophilic coating to the inner surface of the catheter, at step 714. The other is to apply a topcoat to the inner surface of the catheter, at step 716. Similar to the application of the first coat of hydrophilic coating (at step 706) and the application of the basecoat (at step 708), both steps 714 and 716 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. The relevant coating material may dwell within the catheter, rather than flow through, for a predetermined amount of time.

Next, the process continues with placing the catheter back into the oven to dry (or “heat cure”) again, at step 718. Like the first and second drying steps (step 702 and step 710), step 718 may involve placing the catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. The process concludes by disconnecting the positive or negative pressure source and removing the dry, coated catheter from the oven, at step 720.

Similar to FIG. 7, FIG. 8 includes a flowchart illustrating a process of coating and curing an inner surface of a catheter. Unlike the process shown in FIG. 7, which uses heat to cure the coatings inside the catheter, the process of FIG. 8 instead uses ultraviolet (“UV”) light to cure the coatings.

The process shown in FIG. 8 starts with cleaning the catheter, at step 800. In some embodiments, cleaning the catheter includes flushing the catheter with purified water, IPA, a mix of IPA and water, or some other suitable cleansing fluid. The next step is to dry the catheter in an oven, at step 802. The drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. The process continues with step 804: remove the dry catheter from the oven.

Next, the process can continue in one of two possible steps. One option is to apply a first coat of hydrophilic coating to the inner surface of the catheter, shown at step 806. Alternatively, a basecoat may be applied to the inner surface of the catheter, at step 808. Both steps 806 and 808 may use positive or negative pressure to fill the catheter with either the hydrophilic coating (step 806) or the basecoat (step 808). The catheter may be filled with the relevant coating material from either end of the catheter body. In some embodiments, the relevant coating material substantially continuously flows through the catheter for a predetermined amount of time to ensure an adequate amount of coating is applied. The relevant coating material may dwell within the catheter, rather than flow through, for a predetermined amount of time.

After either step 806 or step 808, the process may continue by inserting a UV light apparatus to cure the coating and applying positive or negative pressured air (e.g., oxygen, a mix of oxygen and nitrogen, etc.) to the hub of the catheter in order to dry the inner surface of the catheter, at step 810. In some embodiments, the UV light apparatus is inserted into the inner diameter of the catheter to cure the coating on the inner surface. Next, the positive or negative pressure source is disconnected and the UV light apparatus is removed from the catheter, at step 812.

At this point, the process again diverges into two different options. One is to apply a second coat of hydrophilic coating to the inner surface of the catheter, at step 814. The other is to apply a topcoat to the inner surface of the catheter, at step 816. Similar to the application of the first coat of hydrophilic coating (at step 806) and the application of the basecoat (at step 808), both steps 814 and 816 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. The relevant coating material may dwell within the catheter, rather than flow through, for a predetermined amount of time.

Next, the process continues with another round of UV light curing, at step 818. Like the first UV curing step (step 810), step 818 may involve inserting a UV light apparatus to cure the coating and applying positive or negative pressured air (e.g., oxygen, a mix of oxygen and nitrogen, etc.) to the hub of the catheter in order to dry the inner surface of the catheter. The UV light apparatus may be inserted into the inner diameter of the catheter to cure the coating on the inner surface. The process concludes by disconnecting the positive or negative pressure source and removing the UV light apparatus from the catheter, at step 820.

The catheter 10 may be configured for use in various procedures conducted in various locations of a patient's anatomy. Though brain-specific thrombectomy is discussed, the disclosure should not be considered limiting to any specific type or location of the procedure. The catheter 10 may be used for the aspiration of clots throughout a patient's body, and the various aspects of the catheter 10 discussed above may improve the rate of clot removal in a number of procedure locations.

An example procedure may be conducted using a method comprising inserting the catheter 10 into a patient's vasculature, advancing the catheter 10 through the patient's vasculature toward a vascular lesion, and advancing the catheter to a first location or a second location. In some embodiments, the first location is within a first predetermined distance from the vascular lesion, and the second location is within a second predetermined distance from the vascular lesion. When the catheter 10 is in the first location, the catheter 10 may be able to aspirate the vascular lesion. When the catheter 10 is in the second location, the catheter 10 may be unable to aspirate the vascular lesion. In some embodiments, when the catheter 10 is in the first location, the method further comprises aspirating the vascular lesion with the catheter 10. When the catheter 10 is in the second location, the method may further comprise advancing a secondary device through the catheter 10 toward the first location, as previously discussed in this disclosure. In some embodiments, when the secondary device is in the first location, the method further comprises aspirating the vascular lesion with the secondary device. It should be noted that removal methods other than aspiration may be used with at least one of the catheter 10 and the secondary device.

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are non-limiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and sub-combinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or other ways. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The term “substantially” is used to mean “completely” or “nearly completely.” For example, the disclosure includes, “the first hydrophilic coating comprises a substantially smooth surface.” In this context, “substantially” is used to mean that the first hydrophilic coating may comprise a completely or nearly completely smooth surface.

While certain example embodiments have been described, these embodiments have been presented by way of example only and are not intended to limit the scope of the inventions disclosed herein. Thus, nothing in the preceding description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A catheter, comprising:
a proximal end;
a distal end located opposite the proximal end;
a working lumen extending between the proximal end and the distal end;
an outer surface extending between the proximal end and the distal end, the outer surface defining an outer diameter;
an inner surface extending between the proximal end and the distal end, the inner surface defining an inner diameter, wherein the inner surface is located opposite the outer surface;
a first hydrophilic coating located on the outer surface and configured to reduce surface friction and increase lubricity of the outer surface, the first hydrophilic coating comprising a substantially smooth surface; and
a second hydrophilic coating located on the inner surface and configured to reduce surface friction and increase lubricity of the inner surface, the second hydrophilic coating comprising a textured surface.

2. The catheter of claim 1, wherein the working lumen is configured to receive a secondary device comprising at least one of a guidewire, a microcatheter, an intermediate catheter, and a stent retriever.

3. The catheter of claim 1, further comprising a device wall including at least one polymer coupled to a catheter reinforcement structure.

4. The catheter of claim 3, wherein the at least one polymer is configured to provide at least one of flexibility and structural support to the catheter.

5. The catheter of claim 3, wherein the catheter reinforcement structure comprises a braid and coil reinforcement structure.

6. The catheter of claim 5, wherein the braid and coil reinforcement structure comprises a coil defining a pitch smaller than 0.03 inches.

7. The catheter of claim 3, wherein the device wall is located between the first hydrophilic coating and the second hydrophilic coating.

8. The catheter of claim 1, wherein the first hydrophilic coating extends between the proximal end and the distal end of the catheter.

9. The catheter of claim 1, wherein the first hydrophilic coating extends along a surface extending between the proximal end and the distal end.

10. The catheter of claim 1, wherein the second hydrophilic coating extends between the proximal end and the distal end.

11. The catheter of claim 1, wherein the second hydrophilic coating extends along a surface extending between the proximal end and the distal end.

12. The catheter of claim 1, wherein the outer diameter is about 0.111 inches and the inner diameter is about 0.100 inches.

13. The catheter of claim 1, wherein the outer diameter is about 0.098 inches and the inner diameter is about 0.088 inches.

14. A method of using a catheter, comprising:
inserting a catheter into a patient's vasculature, wherein the catheter includes a proximal end and a distal end located opposite the proximal end;
advancing the catheter through the patient's vasculature toward a vascular lesion; and
advancing the catheter to a location selected from the group consisting of a first location and a second location.

15. The method of claim 14, the first location is within a first predetermined distance from the vascular lesion and the second location is within a second predetermined distance from the vascular lesion.

16. The method of claim 15, wherein when the catheter is in the first location the catheter is able to aspirate the vascular lesion, and when the catheter is in the second location the catheter is unable to aspirate the vascular lesion.

17. The method of claim 16, wherein when the catheter is in the first location, the method further comprising aspirating the vascular lesion with the catheter.

18. The method of claim 17, wherein when the catheter is in the second location the method further comprises advancing a secondary device through the catheter toward the first location.

19. The method of claim 18, wherein when the secondary device is in the first location, the method further comprising aspirating the vascular lesion with the secondary device.

20. The method of claim 14, wherein the vascular lesion comprises an occlusion located in a patient's brain.
